(19) United States
(12) Patent Application Publication
(10) Pub. No.: US 2018/0344635 A1
(43) Pub. Date: Dec. 6, 2018

(54) Instant cubed vitamin packed tablet

(71) Applicant: Chad Galyean, Galesburg, IL (US)

(72) Inventor: Chad Galyean, Galesburg, IL (US)

(21) Appl. No.: 15/614,628

(22) Filed: Jun. 6, 2017

(51) Int. Cl. A61K 9/00; A23L 33/15; A23L 33/16; A23L 33/00

(52) U.S. Cl. CPC A61K 9/0095; A23L 33/15; A23V 2002/00; A23L 33/40; A61K 9/00; A23L 33/16

(57) ABSTRACT

An invention that allows you to enjoy different drinks instantaneously by adding a cubed tablet to liquid. This allows for spontaneous enjoyment of your favorite beverage drink. The drink can be dissolved in hot or cold water with ease by dropping one cube in a cup and stir. This can be used for coffee drinks, chocolate drinks, teas, and instant juices.

### BACKGROUND

### Field of the Invention

The present invention relates to a cubed soluble beverage material. More particularly a water soluble flavored instant cubed vitamin packed tablet beverage.

Aspects of the present invention relate more particularly to the drink that can be dissolved in hot or cold water with ease by dropping one cube in a cup and stir. This can be used for coffee drinks, chocolate drinks, teas, and instant juices.

### Description of the Related Art

A number of processes have been developed for producing instant drinks or dry substances that will dissolve in water to form a desired beverage. Generally, these processes form either granular agglomerates or tablets of the dry beverage substance. The agglomerates are formed by first blending a dry integrated beverage material from substances such as instant coffee, sugar and powdered milk. The material is then ground to a fine particle size, moistened, agitated and dried to form the granular agglomerate.

The common concept is using only a compressing method of particles using only granules. The common method of choice is much different and less effective than that of this current invention. It has been found with this invention that by mixing the ingredients first then allowing them to aerate and then using a combination of a molding and compressing technique followed by heating and cooling the technique gives better results. The integrity of the cubes using this present method makes the cubes more durable than those of past inventions. It has also been found that the end results turn out better when a combination of particles are use including lyophilized coffee, soluble, granules, and dehydrated extracted depending on the preferred drink beverage to be made.

This invention provides for a new way to make an instant beverage with a new makeup can include a combination of different variations of ingredients including granules, soluble, dehydrated ingredients and lyophilized as well as flavorings together with panela then molding and then compressing it make for much better results than the contemporary method. The invention has extracts, panela, granules, soluble, and lyophilized which have high micronutrient content making the cube packed full of vitamins and minerals, antioxidants, and immunological benefits and a low glycerine index which no other invention of the sort currently provides in comparison. With the natural ingredients used the beverage becomes packed with vitamins and minerals which help assist in a number of the bodies functions which is unlike anything else commonly used currently which means this invention gives a health benefit and provides value to anyone using this present invention.

Using this new method of ingredients and preparation allows for a variety of flavors as well as a variety of beverage drinks which was not available in the traditional method of preparation. This invention allows for coffee cubes, tea cubed tablets, chocolate drinks, and juice beverages to be enjoyed instantly. The invention allows for one cube to be dissolved in 8-10 ounces unlike any other invention which would only allow for 4-6 fluid ounces. There is no longer a need to have to have powdered mix which is most commonly used in the market as with the common practice along with no need for any machinery for preparation. With conventional methods to make beverages it makes it hard to measure quantities per services which is not now and you can have measurements already done with accuracy per serving required with the present invention. There is no need to purchase large quantities of a beverage if you would like only one or two beverages with this invention since you can get an individual serving instead of being required to purchase in bulk with past inventions which makes this much more affordable. It is easy to take anyplace any dissolve quickly in fluid allowing for a instantaneous beverage anytime, anywhere and can be made with speed which has not been available with traditional means. The most common practices allow only for dissolution of products with extremely hot fluids. With the submitted invention, it allows for dissolution in both hot or cold fluids which may include water, milk or yogurts. With the ease of matrix manipulation, it also allows that the cubed tablet be available in a number of shapes and sizes which was prevented in the common method of preparation. You will find by using the new method and combination of ingredients the cubed tablet withstands pressure and becomes more durable than other methods previously used. The new method of wrapping then that of previous inventions also allows for a longer lifespan. The current invention can last up to 2 years with the current ingredients and wrapping unlike previous inventions which will last only 6 months to a 1 year maximum. The new invention makes for a longer lifespan of up to 1 year. The present invention is much more durable then past inventions. The cube does not break apart in the wrapping like you find in previous inventions. The new techniques and ingredients combined with the wrapping make for an overall more durable product where previous ones lacked. With the new method, the dissolve time lowers allowing for the beverage to be ready for consumption much quicker than any other previously used method. This invention in its current state is solving the problem by previous inventions of long wait times, lack of variety and durability. The flavored instant cubed vitamin packed tablet beverage is solving problems in the marketplace that current inventions have including long dissolve times, lack of durability, lack of flavors, sizes and shape variety and providing no vitamins and minerals in the product. This invention has solved these problems and the new method to produce the cubes and ingredients makes for a much easier and enjoyable product for the consumer than any of the known contemporary methods and anything in the marketplace. In its current state this invention has shown to improve any previous method for use, previous inventions and previous common practices.

However, to Applicant's knowledge, the cubes formed by previous methods are unsatisfactory. Hence, the inventor of the present invention proposes to resolve and surmount existent difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

According to the first embodiment The invention is regarding a soluble dissolving cubed tablet. The tablet contains vitamins and nutrients. It can be used with hot or cold fluids and dissolved in water or milk. The current invention allows for a juices, teas and coffees as well as chocolate drinks to be consumed instantaneously.

Another object of the invention relates to fill a void from the current self-dissolving products currently available by containing vitamins and minerals, allowing multiple flavors, variety of drinks, a variety of shapes and sizes, a reduced dissolve time, a more durable consistency to withstand crumbling, ease of use, dissolve in 10-35 seconds, 20-55 second dissolve time for stronger condensed cubes, long lasting up to 2 yr lifespan, ability to use in hot or cold drinks and a gourmet flavor.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows the machine for mixing ingredients.

FIG. 2 shows an aerator to sit.

FIG. 3 shows the molding machine.

FIG. 4 shows the molding process which allows the cubes sizes and shapes to be changed.

FIG. 5 shows compacting machine.

FIG. 6 shows the cubes wrapping process.

FIG. 7 shows machine heating and cooling which is the last step.

FIG. 8 shows different sizes and shapes that can be formed.

FIG. 9 shows nutritional data of different vitamins and minerals in the cubes.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

Considering the advances in technology, this invention allows for instantaneous consumption of a beverage. It can be used in hot or cold fluid. The cubes dissolve in milk or water. It allows for teas, coffees, chocolate beverages and juices to be consumed by dropping a cube so it may dissolve and then consume. This invention contains VITAMINS. There is Panela in the mixture which is rich in vitamins and minerals. The cubes will vary in B, A, C, D and E, K vitamins and a number of different minerals as well depending on drink and ingredients. The MINERALS the cubes contain are phosphorous, calcium, iron, magnesium and potassium among many more. The CARBOHYDRATES it contains sucrose, glucose, and fructose Allows for easy accessibility to be used anywhere, anytime and anywhere.

The composition of the cube is made up of a mixture of different ingredients. Panela helps to hold the ingredients together. There are soluble, dehydrated, lyophilized, granules and different extract flavoring ingredients which may be used in the makeup of the cubes depending on which beverage will be made. The most common make up is the coffee cube which will make up of lyophilized coffee, panela, soluble and granules along with different extract flavoring ingredients. When making the teas you will use granules and different dehydrated flavorings along with soluble with extract flavoring to form a tea drink. Dehydrated organic juice powders can be added along with fruit and vegetable and natural plant extracts which combined and compacted in the cube help enrich the vitamins and minerals which the invention contains. The juice cubes will be more have dehydrated extract and flavoring along with some soluble and granules. The ingredients will be mixed slowly gradually adding the flavoring and then left to aerate. The ingredients will be molded and compressed unlike other methods which just compress. Once molded and compressed the cubes will be heated and cooled. This is a new method in making an instant cubed beverage which has completely new ingredients and also allows for a much better product than any other invention to date which brings value to this present invention. Once made each cube will be wrapped individually to allow for a longer lifespan and insuring for a much durable product then current inventions. This invention allows for instantaneous consumption of a beverage and can be used in hot or cold fluid. This flavored instant cubed vitamin packed tablet beverage is unlike any of its kind and improves all other inventions currently in use.

The process for making the cubes starts with gathering the finest quality ingredients. The ingredients are measured to formulation for the desired beverage to be made and put in a bin. Mixing then begins and extract and flavoring is added if necessary for the formulated beverage. The ingredients are left to aerate for an extended period of time. The mixed ingredients are then put into a mold which due to the matrix capability has the ability to change size and shape. The molded ingredients are then compressed at an adequate pressure that has been determined for each needed cube and beverage. The cubes are then put in the wrapping process and then boxed and heated in a thermo heating tunnel and cooled to complete packaging. The process for making the cubes and the ingredients in the cubes make it a unique invention.

Flavoring can include, honey, cappuccino, classic, coffee, black, natural strong coffee, decaffeinated coffee, rum, decaffeinated teas, black teas, hazelnut, vanilla, French vanilla, chocolate, coco, amaretto, baileys, dark teas, whisky, pistachio, mocha, hazelnut, hazelnut cream, vodka, almond, chai, mint, sweetened condensed milk, cinnamon, caramel, thyme, cherry, fruit punch, lemon, orange, passion fruit, blackberry, banana, pineapple, passion fruit, Jasmine, kiwi, grape, peach, strawberry, Sencha, oolong teas, cherry, mandarin, guava, puer, pear, apple, caramel apple, rooibos, papaya, cocoa, mango, tamarind, blueberry, green tea, cardamom, Cranberry, Chamomile, lemon, lime, lemon grass, Chamomile, sweet marjoram, Pomegranate, White teas, Pumpkin, citron, Raspberry, granadilla, Herbal tea, borojo, Carambola (star fruit), toasted marshmallow, butterscotch, peanut butter, cookie dough, chocolate mint, spearmint, peppermint, cheesecake, eggnog, Ginger, Hibiscus, Rose Hips, coconut, lavender, hibiscus, lemonade, limeade, kiwi, watermelon, berry, tangerine, maple, Irish cream, raspberry, feijoa, curuba (banana passion fruit), guanabana, guayaba (guva), lulo, mango, nispero, papaya, pineapple, dragon fruit, cape gooseberry fruit, tree tomato, zapote, mora, maracuya, corozo, caimito, chontodura, nispero, curba, Mamoncillo, Guanaba, Granadilla, pecan, caramel pecan, gingerbread, machadaia. Shapes can include, rectangles cubes, squares, cuboid, circles, ovals, bean shapes, triangle, hearts, trapezium, pentagon, parallelogram, rhombus, diamond, hexagon, hexagon, octagon, nonagon, decagon, stars, cross, arrows, moons, cone, sphere, cylinder, pyramid, prism, box shape.

There are a number of vitamins and minerals in the cubes. The invention is rich in vitamins. The invention is packed with micronutrients.
The MINERALS the cubes contain are phosphorous, calcium, iron, magnesium and potassium among many more.
   The CARBOHYDRATES it contains sucrose, glucose, and fructose Product has immunological benefits
Product has a low glycemic index
Product has a high micronutrient content.
Product has nutrients required by organisms throughout life in small quantities to orchestrate a range of physiological functions
Dehydrated organic juice, vegetable, fruit and plant powders and natural plant extracts.
Grounded graduals and soluble particles.
Liofilozide and dehydrated ingredients
Plant, fruit and vegetable extracts.
Natural and artificial flavoring.

The size of the tablets can be modified. This is because the matrix of the machine can be adjusted and the composite of the ingredients and the process in which the cubes are made will allow for a change in the structure without losing any of its integrity. Changing the sizes allows for different shapes and dimensions. Some of the most common used are 8 cm×3 cm×5 cm, 3 cm×2 cm×3 cm, 10 cm×6.5×1.5 cm, 10.5×7.5×2 cm, 11×5×3 cm, 8 cm×10×2.5, 11.5×6.0×1.75, 12.1×4.5×4.5 cm, 2.75 cm×2.75 cm×0.75 cm. Sizes and shapes may vary depending on the matrix modifications. There can be a number of different weights for the cubed tableted beverage the following are some examples 8 grams, 7 grams, 12 grams, 18 grams, 4 grams, 5 grams, 9 grams 20, 25 grams, grams 14.5, grams. Weights may vary depending on size and shape of cubes. The percentages of ingredients used will change according to which drink will be used and according to how strong the drink will be made. The following percentages are examples of most commonly used percentages. Panela 20 %, ingredients 75 %, flavoring 5 %, 59.25 %panela, coffee 37.50 %, flavor 3.25 %, panela 39.00 %, ingredients 59 %, ingredients 2 %. Percentages can vary depending on the size and shape, strength of the cube, and the beverage and ingredients being used.

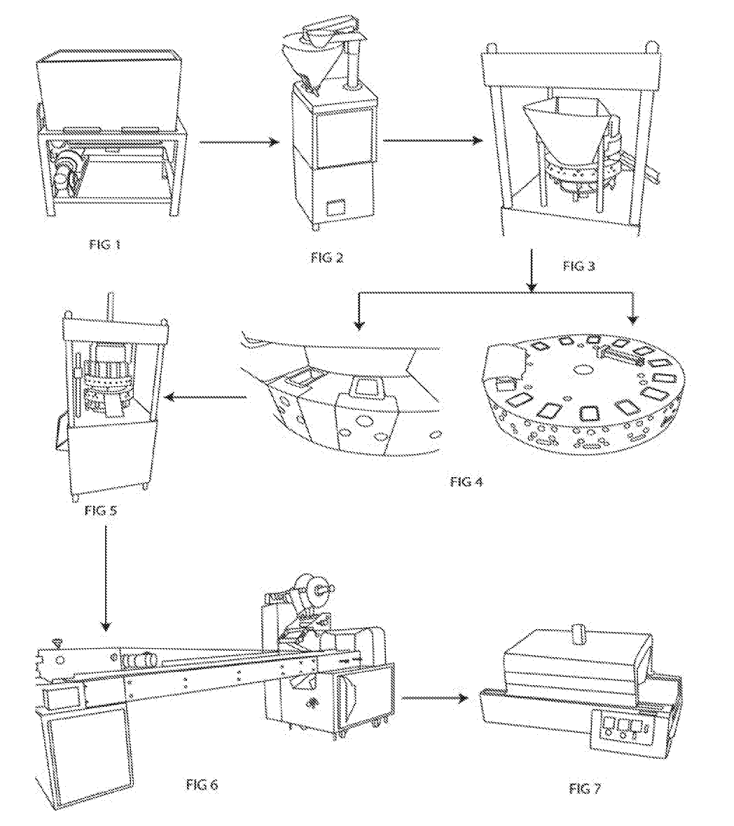

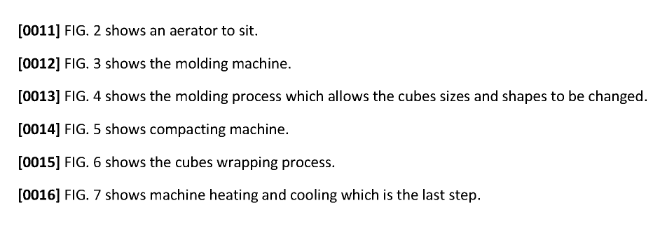

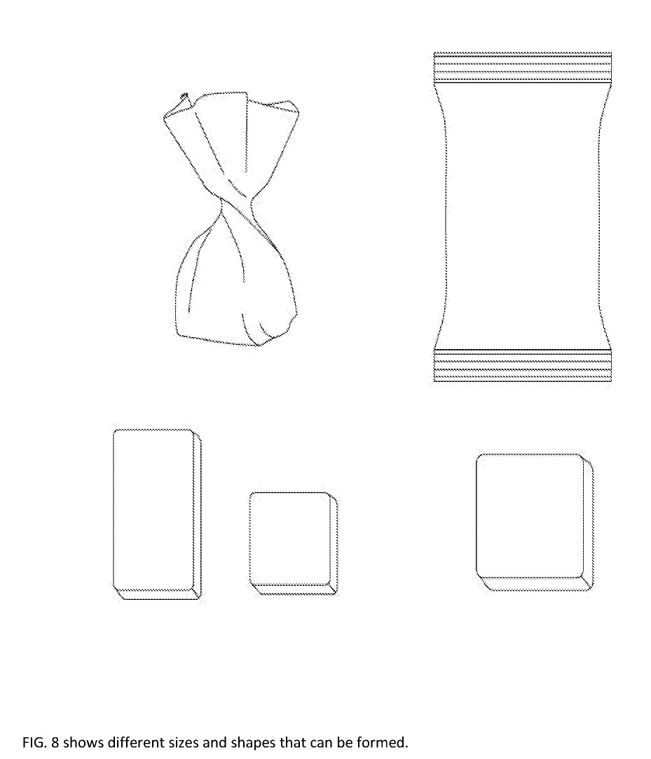

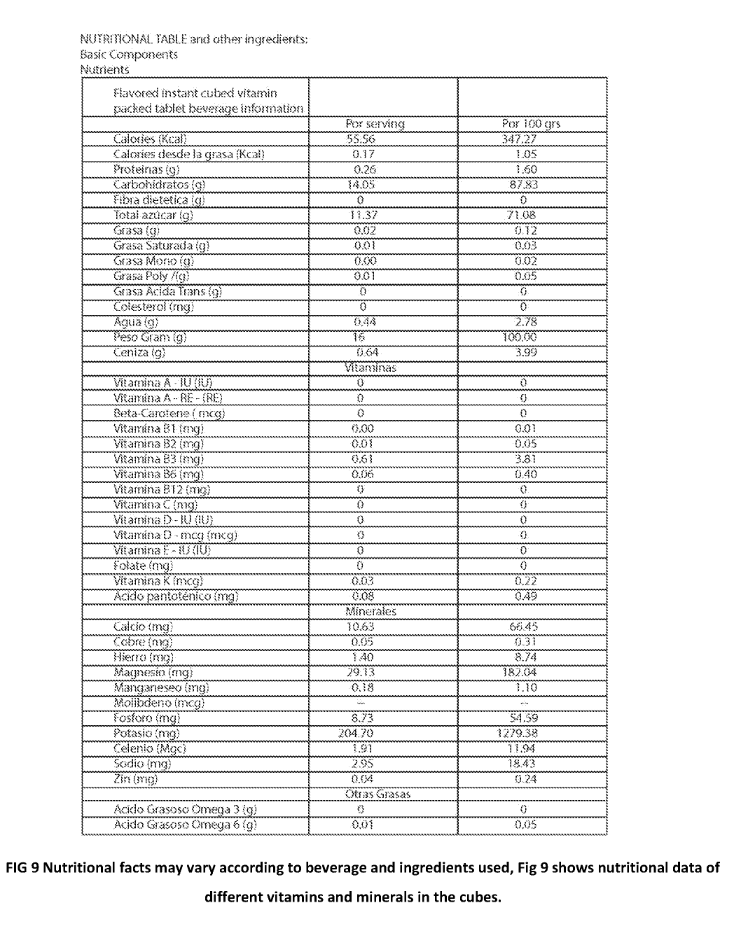

FIG. 1-7 shows flowchart process of the cubes that are made. FIG. 1 one show how the ingredients are gathered that are to be mixed in the machine. FIG. 2 shows after the mixing process the ingredients are put in an aerator to sit. FIG. 3 shows how the ingredients are then molded in the machine. 4 shows the matrix used in the molding process and it is because of the ingredients used as well as it allows the cubes sizes and. shapes to be changed. FIG. 5 shows how they are compacted. FIG. 6 shows how the cubes are wrapped. FIG. 7 shows how cubes are heated and cooled after they are made which completes the whole new process. FIG. 8 shows examples of the cubes that can be made from the process. The sizes and shapes may vary according to the changing of the matrix. FIG. 9 shows the different vitamins and minerals in the cubes. This nutritional data may vary according to the beverage drink being made and the cubes ingredients being used.

Having described the invention in detail, those skilled in the art will appreciate that modifications may be made to the invention without departing from its spirit.

1. The present invention is a water soluble flavored instant cubed vitamin packed tablet beverage comprising:
a) Panela a component healthier than sugar.
b) Cube which dissolves in 10-35 seconds and can be mixed in water or milk.
c) The cubes are rich in vitamins and minerals and contain micronutrients.
d) Nutrients required by organisms throughout life in small quantities to orchestrate a range of physiological functions.

2. Cube which dissolves in 10-35 seconds and can be mixed in water or milk whereas stronger condensed cubes can take 20-55 seconds dissolve time.
f) Can dissolve in fluids including milk, water and yogurts.
